(19) United States
(12) Patent Application Publication
(10) Pub. No.: US 2025/0231186 A1
(43) Pub. Date: Jul. 17, 2025

(54) COUPLING ASSAY FOR T CELL SPECIFICITY (CATS) AND METHOD OF ITS USE

(71) Applicant: ARIZONA BOARD OF REGENTS ON BEHALF OF THE UNIVERSITY OF ARIZONA, Tucson, AZ (US)

(72) Inventors: Michael KUHNS, Tucson, AZ (US); Bradley Pace MOFFETT, Tucson, AZ (US); Caleb KIM, Tucson, AZ (US); Mark LEE, Tucson, AZ (US)

(21) Appl. No.: 18/856,648

(22) PCT Filed: Apr. 20, 2023
(86) PCT No.: PCT/US2023/019268

§ 371 Date: Oct. 14, 2024

Related U.S. Application Data
(60) Provisional application No. 63/363,263, filed on Apr. 20, 2022.

(51) Int. Cl. G01N 33/569; G01N 33/68

(52) U.S. Cl. CPC G01N 33/56972; G01N 33/6845; G01N 2333/7051

(57) ABSTRACT

A technique, called the Coupling Assay for T-cell Specificity (CATS), to identify antigen-specific cells using cell lines expressing MHC II molecules with tethered peptides. CATS successfully identified antigen-specific T cells with a low-affinity peptide, while tetramer failed to identify cells with this same peptide. Increasing avidity on artificial antigen presenting cells can overcome low affinity TCR-pMHC interactions, can identify more responding endogenous populations, and may be specific for the MHCII.

### RELATED APPLICATION

This application claims priority to U.S. Provisional Application 63/363,263 filed on Apr. 20, 2022, the content of which is incorporated herein by reference in its entireties for all purposes.

### GOVERNMENT RIGHTS

This invention was made with government support under Grant No. AI101053 awarded by National Institutes of Health. The government has certain rights in the invention.

### BACKGROUND

T cells are a special type of cells that mediate our adaptive immune responses. When microbes enter our body, dendritic cells, natural killer cells (NK cells), macrophages, and neutrophils act as our first line of defense. These cells mediate our innate immune response, which rely on pattern recognition receptors (PRRs) to identify and eliminate harmful microbes that display pathogen-associated molecular patterns (PAMPs) (Morgensen, 2009). Infection can also trigger our adaptive immune response, which is carried out by T cells and B cells. There are several types of T cells, including CD8+ and CD4+ T cells, that each perform different functions, such as killing harmful cells or increasing cytokine production in helper T cells (Li et al., 2013). Together, our innate and adaptive immune responses work in tandem to defend us from lethal infections and invasions.

T cells use their T cell receptors (TCRs) to survey peptide antigens attached to class I or class II major histocompatibility complex (pMHC-I or pMHC-II) molecules on antigen-presenting cells (APCs) (Kuhns et al. 2012). T cells are highly specific and unique, possessing a large repertoire of TCRs which allow them to be selective when scanning across APCs (Moon et al., 2007). When a microbe enters the body, it will first encounter the innate immune response, which may result in the microbe being taken in by a macrophage, degraded into pieces, and presented in fragments on the cell surface using MHC molecules. Unique TCRs are then able to scan and interact with the MHC molecules that now display a peptide. If the binding affinity between the TCR and the pMHC molecule is high enough to induce signaling, the T cell will be activated to help identify and clear out infections in the short term. In order to ward off a similar infection in the future, activated T cells can develop into memory T cells and provide long term immunity.

There are several assays designed to characterize T cell responses, including cytokine production assays, limited dilution proliferation assays, and antigen-specific T-cell targeting. The latter describes a process in which TCRs are targeted with specific pMHC molecules which they bind strongly to, allowing researchers to focus solely on a particular T cell population. However, many T cell-pMHC interactions are weak in order to allow the T cell to disengage, which renders these interactions difficult to observe (Martinez, 2016).

One approach to solving this issue came through the creation of a tetramer molecule, which contains four biotinylated monomeric pMHC molecules that are bound together using streptavidin (Altman et al., 1996). This biotinylated tetramer molecule binds onto multiple TCRs on a T cell, thus extending the half-life of the interaction and allowing scientists to observe some of these interactions (Corse et. al, 2010). The K_{D} of biotin and streptavidin is 10⁻¹⁵ M, which indicates a high affinity for one another. These streptavidin proteins are also conjugated to a fluorescent protein so that tetramer engagement to a T cell may be measured on a flow cytometer.

The benefit of tetramers was noticed immediately: T cell-pMHC interactions were suddenly possible to observe because tetramers elevated the overall affinity, or avidity, of the T cell to the pMHC interactions (Altman et al., 1996). This paper describes a process where this four-pronged molecule can engage more than one TCR at once, which not only holds the molecules together for longer, but it may double or even triple the likelihood that weaker TCR-pMHC interactions will form.

Although tetramers can effectively identify strong TCR-pMHC interactions with great specificity, weaker TCR-pMHC interactions are still difficult to identify. Previous literature suggests that tetramer can only identify between 5-50% of all TCRs that are reactive to a particular pMHC (Huang et al. 2016; Martinez et al., 2016). This poses an issue to the scientific community, especially because many TCR-pMHC interactions are mediated through weak binding in order to send a signal and then break from the molecule of interest (Matsui et al., 1991). These very weak interactions may very well be crucial to the function of our immune response, yet we are unable to study them in depth with the current technology (Martinez et al., 2016). Furthermore, generating tetramers is costly and also sometimes not possible. For instance, there are three immunodominant West Nile Virus (WNV) epitopes that have been identified in mouse MHCII I-A^{b}, however we have only been able to create pMHCII monomers with one of these epitopes (E641) (Deshpande et al., 2015). This incapability limits in our ability to study WNV-specific CD4+ T cells and also makes it costly to generate these monomers.

### SUMMARY

One objective of this disclosure is to develop a novel technology for identifying antigen-specific T cells that presents a more affordable and sensitive alternative to tetramers. This novel assay, coined the Coupling Assay for T cell Specificity (CATS), will be used to determine the specificity, sensitivity, and ability to identify T cell responses when compared to tetramer analysis.

In one embodiment, M12 and 58α⁻β⁻ hybridoma cell lines expressing MHC molecules with tethered peptide on the order of approximately 10⁵ are generated, a significant increase from 4 molecules. If these cells were to be coupled with antigen-specific T cells, the relative avidity for one another should be significantly higher than that with tetramers. The greatest implication of this innovation is that higher avidity binding will allow weaker TCR-pMHC interactions to be observed while also providing a useful tool to further our knowledge of T cells.

Disclosed here are methods to examine the specificity of CATS to identify antigen-specific T cells. In one embodiment, MHCII+ M12 cells with tethered peptides that each have different affinities for 5c.c7 TCR are used to determine T cell activation. These peptides are MCC 88-103 (K_{D}=20-43 μM), the altered peptide ligand T102S (K_{D}≥206 μM) and T102G (K_{D}=too weak to measure by Surface Plasmon Resonance), and the null Hb 64-76 (Huppa et al., 2010). Also disclosed are various ways CATS may be optimized in order to produce a viable method that other labs may use. Next, the sensitivity of CATS are also explored by measuring its ability to identify adoptively transferred 5c.c7 CD4+ T cells within a polyclonal B10.A cell population and comparing it to tetramer technology. It is demonstrated here the ability for CATS to identify weak TCR-pMHC-interactions where tetramers fall short. Lastly, we show the ability of CATS to identify naïve CD4+ T cells in polyclonal population and determine if this assay is dependent on MHC-II.

Ultimately, this disclosure presents an alternative strategy to tetramers to identify antigen-specific T cells that is more sensitive, more affordable, and more accessible to researchers.

In some embodiments, a system called Coupling Assay for T cell Specificity (CATS) is disclosed for detecting activation of T cell receptor (TCR). The system comprises a plurality of cells expressing an MHC molecule and a peptide, wherein the peptide is tethered to the MHC to form an MHC-peptide complex. In one aspect, the MHC-peptide complex is presented on surface of the plurality of cells, wherein the copy number of the MHC-peptide complex ranges between 10³ and 10⁷, or between 10⁴ and 10⁶ or about 10⁵ per cell.

In another aspect, the MHC molecule is a MHC class II molecule (MHCII). In another aspect, the plurality of cells is derived from a cell line that is capable of perpetuating indefinitely.

In some embodiments, the plurality of cells is derived from a lymphoma cell line. In one aspect, the plurality of cells is derived from M12 cell line. In another aspect, the M12 cells express full-length I-E^{k} MHC.

In some embodiments, the peptide is derived from a foreign pathogen, for example, from a bacterium, or a virus. In some embodiments, the peptide is MCC protein (Cytochrome C from Moth).

In some embodiments, the peptide is derived from a tumor cell and the MHC is an MHC class I and the system may be used to fight cancer by activating T cells.

In some embodiments, the peptide is selected from a library comprising a plurality of peptides that are randomly synthesized. In one aspect, these synthesized peptides form a library that can be used to detect and screen for activated T cells.

In some embodiments, the peptide is 10-50 amino acids long, or 10-20amino acids long, or 12-18 amino acids long.

In some embodiments, the system may further comprise a second cell, the second cell comprising a TCR. In one aspect, the TCR of the second cell binds to the MHCII-peptide. In some embodiments, the K_{D} between the TCR and the peptide is greater than 5×10⁻⁶M, greater than 10⁻⁶M, or greater than 5×10⁻⁵M, or greater than 10⁻⁵M, or greater than 5×10⁻⁴M. In another aspect, the second cell is a T cell line. In another aspect, the second cell is a primary CD4 T cell. In another aspect, the second cell is 58α⁻β⁻ cell.

In some embodiments, the peptide is tethered to the MHCII through a linker. In one aspect, the linker comprises a sequence of SGGGGS. In another aspect, the linker comprises a sequence of AAAGGGGSGGGGSGGGGS.

In some embodiments, a method for detecting activation of a T cell receptor (TCR), the method comprising (a) contacting a T cell comprising a TCR with a plurality of cells expressing an MHC molecule and a peptide, and (b) determining association between the T cell and the plurality of cells expressing the MHC molecule and the peptide, wherein the peptide is tethered to the MHC to form an MHC-peptide complex, the MHC-peptide complex being presented on surface of said cell, wherein the copy number of the MHC-peptide complex ranges between 10³ and 10⁷, or between 10⁴ and 10⁶ or about 10⁵ per cell. In one aspect, step (b) is performed by flow cytometry. In another aspect, the T cell and the plurality of cells expressing the MHC molecule and the peptide are labeled by different dyes.

### BRIEF DESCRIPTION OF THE DRAWINGS

FIG. 1A shows Representative flow plots showing dye-labeled 5c.c7 TCRαβG (GFP) TCR 58α⁻β⁻ cells coupled to specific APC dye-labeled pMHC+ M12 cells. Peplide-dependent coupling percentages and known Ko values are shown.

FIG. 2A shows Representative flow plot showing 5c.c7 CD4⁺ T cells coupled with tethered pMHC-II expressing M12 cells. FIG. 2B shows Coupling percentage of T cells to MCC M12 cells coupled 1:1:1 T cell:MCC:Hb at various time points with PP2 kinase inhibitor or DMSO control. FIG. 2C shows Same process as 2B with cells coupled 1:2:2 T cell:MCC:Hb. Statistical analysis was performed using multiple t-test comparison with Holm-Sidak post-test between the average of 3 experiments of PP2 and DMSO treated samples with SEM error bars shown. Significant p values are shown.

FIG. 3 shows results when 1×10⁵ dye-labeled 5c.c7 CD4⁺ T cells are adoptively transferred into a B1 O.A recipient mouse. After 24 hours, spleen and lymph nodes were collected and CATS or tetramer analysis was performed. (A) Representative flow plots showing 5c.c7 CD4+ T cells to specific pMHC-II+M12 cells. (B) Representative flow plots showing 5c.c7 CD4+ T cells stained with specific tetramer in two-color. (C) MCC or T102S cell coupling or tetramer staining percentage to total dye-labeled T cells. Statistical analysis was performed using an ordinary one-way ANOVA with Sidak post-test comparing each category to each other. Exact p values are shown. (n=8 mice).

FIG. 4A shows Representative flow plots showing dye-labeled B1 0.A CD4+ T cells coupled with tethered pMHC-II expressing M12 cells. 4B shows Representative flow plots showing the coupling in unblocked, stained with isotype control antibody, or blocked with aMHC-II antibody states. 4C shows Representative flow plot showing 20 μg/mL 14-4-4S anti-I-E^{k} antibody blocking MHCII I-E^{k} epitopes. 4D shows Relative coupling rates of B10.A CD4+ T cells with tethered pMHC-II expressing M12 cells normalized to blocked states. Representative of 1 experiment.

### DETAILED DESCRIPTION

T cells express TCRs that can interact with pMHC molecules in response to microbial infection. Detecting this interaction is important to understanding T cells. There are several techniques that can characterize a T cell response to infection, including antigen-specific T cell targeting. One such approach utilizes tetramers, a four-pronged pMHC molecule that has been used to target antigen-specific T cells. While extremely useful, tetramers possess limitations, as they are oftentimes difficult and costly to make.

CATS disclosed here offers a viable alternative to tetramer generation. Utilizing B or T cell lymphomas, cell lines are generated expressing pMHCII molecules with tethered peptide. These cell lines were used to target 58α⁻β⁻ cells expressing 5c.c7 TCR, 5c.c7 CD4+ T cells, and naïve B10.A CD4+ T cells to better understand the capabilities and limitations of the CATS assay. Because tetramers were successful at identifying antigen-specific T cells with great specificity, we expected our M12 pMHC+ cell lines to be even more effective at detecting strong and weak TCR-pMHC interactions.

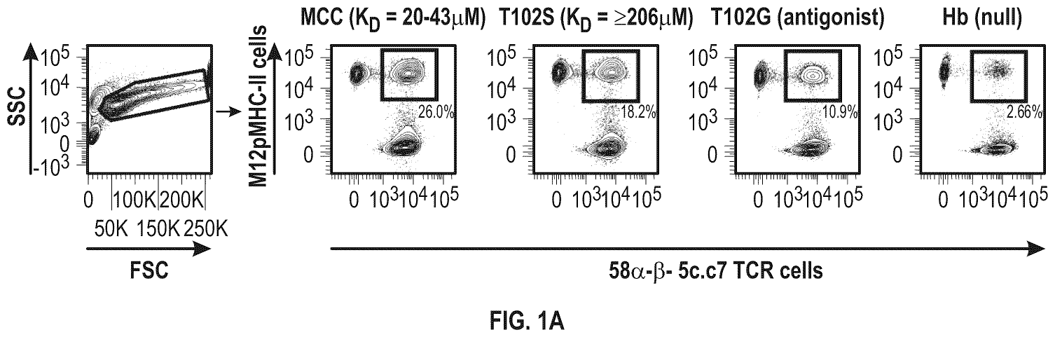

The data presented in FIG. 1 highlight the ability of CATS to identify TCR-pMHC interactions when 5c.c7 TCR+ 58α⁻β⁻ cells were coupled to pMHC+ M12 cells with tethered peptides of varying affinity for the 5c.c7 TCR. The trend we witness complies with our understanding of peptide affinity, with coupling percentage decreasing from MCC>T102S>T102G>Hb. Something to note is that we detected antigen-specific interactions data above background levels in the antagonist T102G, whose K_{D} is too weak to measure via SPR. This suggests that CATS may overcome differences in affinity for peptide by increasing the avidity of the interaction.

Various parameters of CATS assay were altered to determine whether incubation time, ratio of T cell:specific APC:dump APC, or introduction of a kinase inhibitor influences coupling percentage. We concluded that incubating the cell couples at 37° C. ultimately resulted in a decreased percentage of cell couples when the kinase inhibitor PP2 was not present. In fact, when PP2 was present, the coupling percentage appeared to trend slightly upward over time. Next, we observed a negligible shift in coupling percentage between a 1:1:1 and 1:2:2 ratio, indicating that a 1:1:1 ratio is sufficient for the purposes of this experiment. It is also determined that 2 minutes of incubation time provided the highest rate of coupling and is sufficient for future experiments. This factor could greatly reduce the waiting period before cell couples can be analyzed, especially since co-cultures and tetramer staining can take hours until the sample is ready to by analyzed.

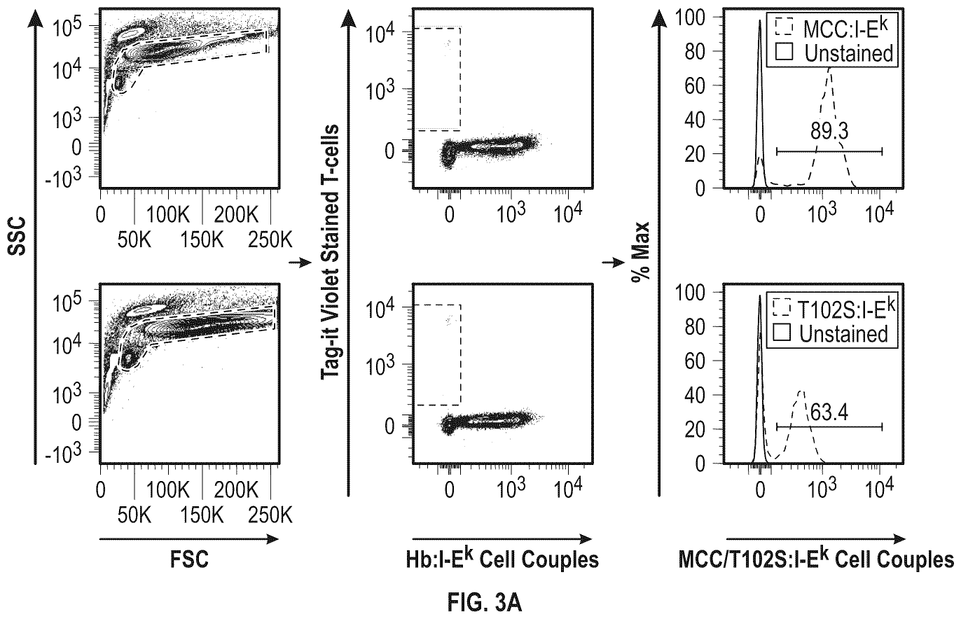

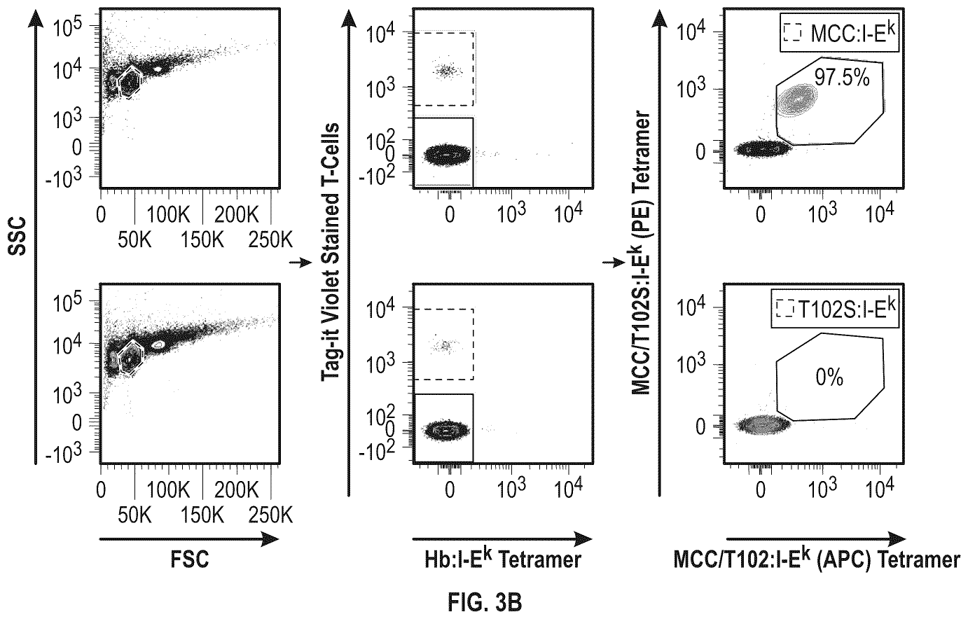

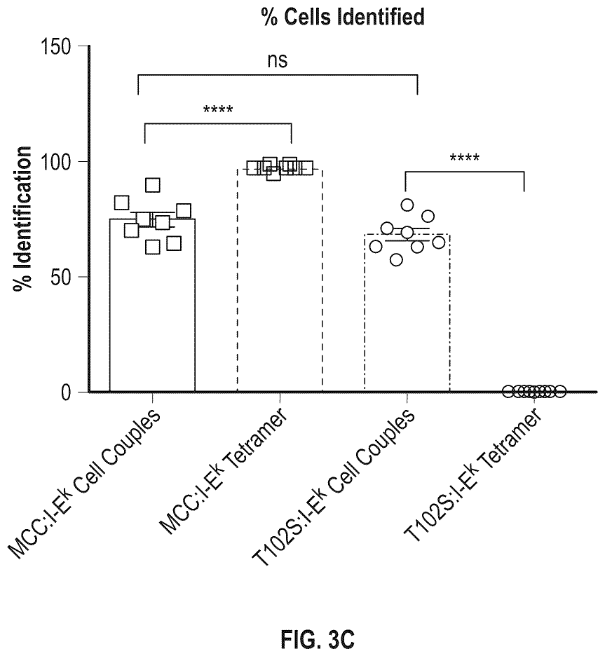

Beyond establishing CATS and optimizing the parameters within it, the primary goal of this study is to compare CATS to tetramer. To approach this question, we measured them head-to-head in FIG. 3 to determine how well each method identified 5c.c7 CD4+ T cells within a polyclonal B10.A mouse. CATS was performed as determined by the optimal methods described above, and tetramer staining was mimicked after CATS by including a dump tetramer. However, for added security, we created the tetramer in two-color to ensure that no cells were randomly sticking to tetramer or auto fluorescing. We discovered that the T102S tetramer does not stain 5c.c7 CD4+ T cells with much success at all. However, we did notice that T102S tetramer could stain the polyclonal B10.A population to a certain extent. Because of this quality, we are certain that the tetramers were not compromised or folding incorrectly prior to engagement. CATS also utilized T102S peptide, however, we noticed a significant shift in 5c.c7 CD4+ T cell identification from 0% with tetramer to around 70% with the cell line. This confirms our prediction that increasing the relative avidity of the TCR-pMHC interactions can result in a higher frequency of identified antigen-specific T cells.

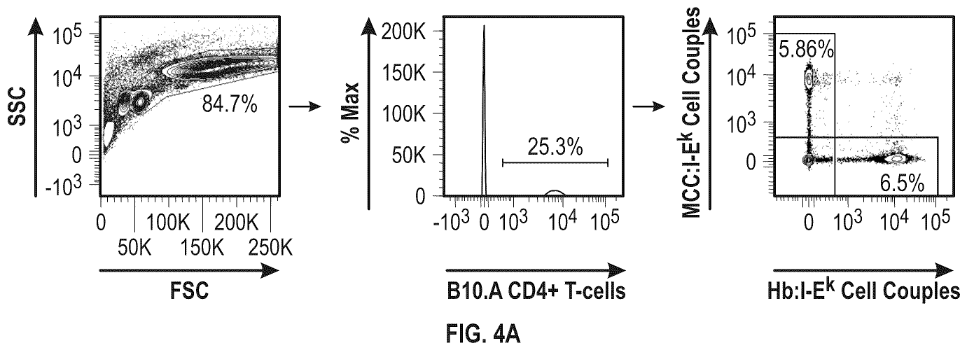

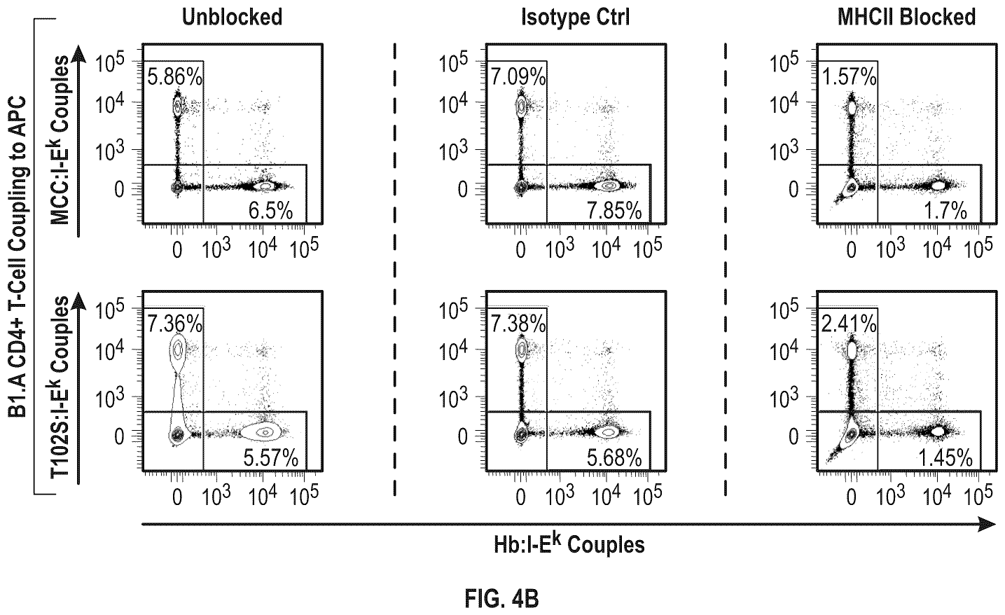

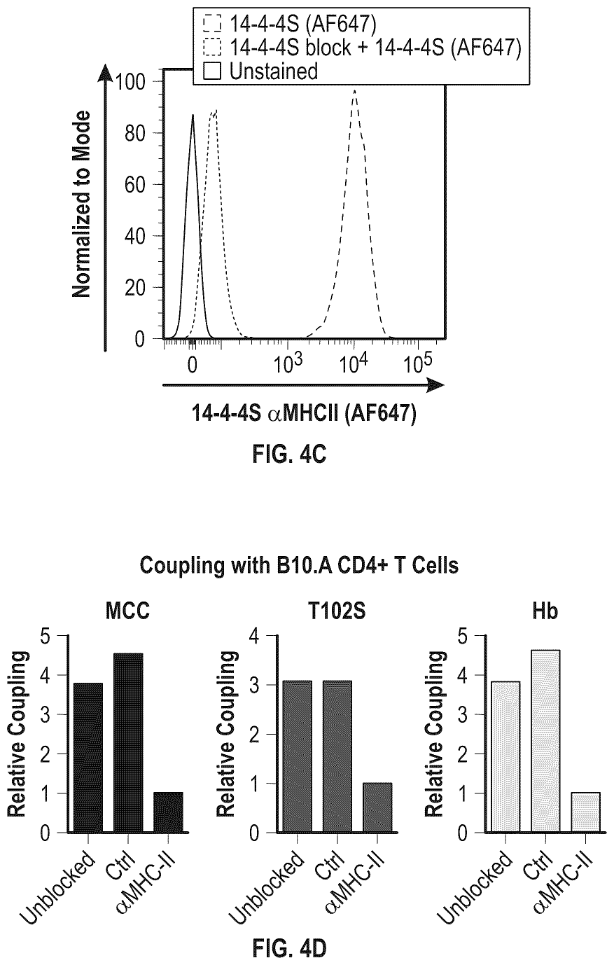

It is disclosed here that CATS is a useful tool to identify antigen-specific T cells, particularly when confronted with the obstacles of low-affinity peptide interactions that tetramers face. However, our next set of questions address cell populations that include the 5c.c7 TCR as well as many others within the TCR repertoire that exist in a polyclonal population. For the purposes of our experiment, we collected B10.A spleens and lymphocytes and conducted CATS on its CD4+ T cells. This experiment was targeted to determine how much the interaction of MHC-II with these T cells affected cell coupling, and hence, identification of endogenous B10.A CD4+ T cells. We predicted coupling to decrease with the addition of clone 14-4-4S (anti-I-E^{k}) as IL-2 production has previously been shown to decrease after MHC-II blocking of pMHC+ M12 cells prior to cell coupling (Parrish et al. 2016). In FIG. 4, we determined that blocking with the 14-4-4S clone was successful at inhibiting TCR-pMHC interactions, as relative coupling in the blocked states were lower by a factor of 3-5× compared to the uncoupled and isotype control states. Although this was a pilot experiment, it provides promising data that MHC-II may play a critical role in CATS, especially when engaging endogenous polyclonal populations.

Despite the success we have had with CATS, there are a few factors we believe are necessary to explore more in-depth. First, our optimization of CATS implements three factors that we deemed critical to explore. However, testing CATS at further ratios and more time points could offer a more robust procedure with higher rates of coupling. Another factor that warrants further exploration is leaving cells on ice and staining with antibody. Initially, we conducted this assay at room temperature and avoided staining with antibody to preserve cell couples. However, if we can stain with antibody on ice, this can open the door to any number of possibilities to employ with CATS.

Because naïve CD4+ T cells possess a large repertoire of TCRs, we learned that our MHCII blocking experiment lacks a true dump cell line, as there are approximately as many TCRs that can recognize Hb as can recognize MCC. One way to approach this problem is by switching to 58α⁻β⁻ generated cell lines and use 58α⁻β⁻ parentals as a dump. This may produce a lower background than M12 parentals, as there is no endogenous class II MHC on 58α⁻β⁻ cells, while there is on M12 cells. Another approach to this question can utilize M12 cells expressing full-length I-E^{k} with no tethered peptide. These cells express several peptides in the MHC-II binding groove, therefore decreasing the avidity of a specific peptide for an antigen-specific T cell. In either case, these controls can better address whether CATS performed on endogenous polyclonal populations is a product of peptide or MHC-II specificity.

The present disclosure is further illustrated by the following embodiments:
Item 1. A system for detecting activation of T cell receptor (TCR), comprising a plurality of cells expressing a major histocompatibility complex (MHC) molecule and a peptide, wherein the peptide is tethered to the MHC to form an MHC-peptide complex, the MHC-peptide complex being presented on surface of said cell, wherein the copy number of the MHC-peptide complex ranges between 10³ and 10⁷, or between 10⁴ and 10⁶ or about 10⁵ per cell.
Item 2. The system of Item 1, wherein the MHC molecule is an MHC class II molecule (MHCII).
Item 3. The system of any preceding Items, wherein the plurality of cells is derived from a cell line that is capable of perpetuating indefinitely.
Item 4. The system of any preceding Items, wherein the plurality of cells is derived from a lymphoma cell line.
Item 5. The system of any preceding Items, wherein the plurality of cells is derived from M12 cells.
Item 6. The system of any preceding Items, wherein the plurality of cells is M12 cells expressing full-length I-E^{k} MHC.
Item 7. The system of any of Items 2-6, wherein the peptide is derived from a foreign pathogen.
Item 8. The system of Item 1, wherein the peptide is derived from a tumor cell and the MHC is an MHC class I.
Item 9. The system of any preceding Items, wherein the peptide is selected from a library comprising a plurality of peptides that are randomly synthesized.
Item 10. The system of any preceding Items, wherein the peptide is 10-50 amino acids long, or 10-20 amino acids long, or 12-18 amino acids long.
Item 11. The system of any preceding Items, further comprising a second cell, the second cell comprising a TCR.
Item 12. The system of any preceding Items, wherein the second cell is 58α⁻β⁻ cell or a primary CD4 T cell.
Item 13. The system of any preceding Items, wherein the K_{D} between the TCR and the peptide is greater than 10⁻⁶M, or greater than 10⁻⁵M.
Item 14. The system of any preceding Items, wherein the peptide is tethered to the MHCII through a linker comprising the sequence of S-G-G-G-G-S.
Item 15. A method for detecting activation of a T cell receptor (TCR), the method comprising (a) contacting a T cell comprising a TCR with a plurality of cells expressing an MHC molecule and a peptide, and (b) determining association between the T cell and the plurality of cells expressing the MHC molecule and the peptide, wherein the peptide is tethered to the MHC to form an MHC-peptide complex, the MHC-peptide complex being presented on surface of said cell, wherein the copy number of the MHC-peptide complex ranges between 10³ and 10⁷, or between 10⁴ and 10⁶ or about 10⁵ per cell.
Item 16. The method of any Item 15, wherein step (b) is performed by flow cytometry.
Item 17. The method of any of Items 15-16, wherein the T cell and the plurality of cells expressing the MHC molecule and the peptide are labeled by different dyes.

### EXAMPLES

The disclosure will now be illustrated with working examples, which are intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 Materials and Methods

### Mice

6-to 8-week-old male and female 5c.c7 TCR Rag KO and B10.A mice were used for spleenocyte and lymphocyte cell coupling and tetramer staining. Mice were maintained under specific pathogen-free conditions in the University of Arizona animal facility. Experiments were conducted under the guidelines and approval of the University of Arizona Institutional Animal Care and Use Committee.

### Cell Lines

58α⁻β⁻ and M12 cells were generated by retroviral transduction using the MSCV-based retroviral expression vectors pP2 (IRES-puromycin resistance) and pZ4 (IRES-zeocin resistance) (Glassman et al., 2016; Lee et al., 2015; Parrishet al., 2016).

The 58α⁻β⁻ cell lines were retrovirally transduced to express 5c.c7 TCR, full-length CD3 subunits, and C-terminally truncated CD4 (CD4T aa:1-421) (Glassman et al., 2016). The C-terminus of the 5c.c7 a chain was fused to mEGFP via a long flexible linker (AAAGGGGSGGGGSGGGGS). The 5c.c7 B chain and CD4T were encoded by independent constructs and full-length CD3 subunits were encoded by a poly-cistronic construct as previously described (Glassman et al., 2016; Parrish et al., 2016).

M12 lines were generated by transducing M12 parental cells with full-length I-E^{k}α and full-length I-E^{k}β, fused at the N-terminus to a peptide as previously described (Parrish et al., 2016; Parrish et al., 2015). The peptides in this study include moth cytochrome c peptide (MCC) 88-103 (ANERADLIAYLKQATK), the altered peptide ligands of MCC, T102S and T102G, and the mouse hemoglobin d allele Hb 64-76 (GKKVITAFNEGLK).

Cell surface expression of CD4, TCRα, TCRβ, and I-E^{k} were determined by flow cytometry as previously shown (Glassman et al. 2016).

### Lymph Node and Spleen Dissection and Dissociation

Inguinal, brachial, and axillary lymph nodes (LN) and spleens were collected from mice. They were dissociated using frosted glass cover slides and treated with Ack lysing buffer before being resuspended in RPMI.

### CD4+ T-Cell Enrichment

T-cells were counted using the Hemavet instrument. Miltenyi CD4+ cell isolation kits were used in conjunction with Miltenyi LD columns and MACS magnetic separators to enrich the CD4+ T-cell population. Cells were spun down and resuspended in 40 μL of complete RPMI per 10⁷ cells. Next, 10 μL per 10⁷ cells of CD4+ antibody cocktail was added to the solution, mixed thoroughly, and left on ice for 5 minutes. In that time, 3 mL complete RPMI was flowed through the LD columns on the separators. 30 μL per 10⁷ cells of complete RPMI was added to the tube after the 5-minute stain period. Finally, 20 μL per 10⁷ cells of anti-biotin microbeads was added to the tube, mixed thoroughly, and left on ice for 10 minutes. After the waiting period, 3 mL of complete RPMI was added to the tube and the contents were transported into the MACS column. Flow through (CD4+ T cells) was collected. Pre- and post-enrichment analysis was conducted via Flow Cytometry.

### Adoptive Transfer

1×10⁵ Tag-it Violet stained 5c.c7 T cells in 100 μL of PBS were retro-orbitally injected into a B10.A mouse. After 24 hours, LN and spleen were collected for analysis.

### Cell Membrane Staining

Cells were counted, resuspended in 5×10⁶ cells/ml of 0.2% FBS PBS and cell surface stain dye. 1 μL of 5 mM Tag it Violet, Cell Trace Far Red, or Cell Trace CFSE dye was added per 1 mL of 0.2% FBS PBS, for a final concentration of 5 μM, as described by the manufacturer. Cells were mixed and incubated at 37° C. for 20 minutes. After the waiting period, 5 ml of complete RPMI was added to the sample to quench any remaining dye.

### Cell Coupling Assay

Either TCR+ CD4+ T cell hybridomas or CD4+ T cells from mice were coupled with M12 cells expressing pMHC class II at a 1:1:1 ratio (T-cell:specific APC:dump APC), spun down for 5 minutes at 1500 RPM and incubated at 37° C. for 2 minutes. Cells were washed with 2% FBS PBS and immediately prepared to flow.

### Tetramer Preparation

pMHC monomer was added to conjugated streptavidin at a ratio of 4:1 and 2% FBS PBS was added to achieve the final concentration of 4 μM:1 μM. The total concentration was further diluted into the cell population.

### Tetramer Analysis

T cells expressing TCR were spun down and resuspended in 300 μL 24.G2 FC Block (with 0.002% azide+2% mouse serum) and incubated for 20 minutes on ice. Cells were washed with 2% FBS PBS and resuspended in 190 μL. 5L of tetramer was added to each tube for a total volume of 200 μL. Cells were mixed thoroughly and allowed to stain overnight at 4° C. This results in a total tetramer concentration of 100 nM:25 nM monomer:streptavidin.

### Flow Cytometry

BD LSR II and BD Fortessa measured cell engagement to CATS or tetramer

### Example 2 Demonstration and Optimization of CATS

The first goal was to develop a working assay that can be used to demonstrate CATS' utility. Initially, CATS was performed by coupling 5c.c7 TCR+ 58α⁻β⁻ cells with Tag-it Violet labeled pMHC+ M12 cells at a ratio of 1:1 and incubated couples at 37° C. for time periods of 0 minutes, 2 minutes, 20 minutes, and 60 minutes. Flow cytometry was used to exclude single populations of 58α⁻β⁻ or M12 cells, gating on the double positive population (FIG. 1A). In this experiment, we used 4 distinct peptides tethered to MHCII molecules, all with affinity for 5c.c7 TCR as follows: MCC>T102S>T102G>Hb. MCC represents the cognate peptide for 5c.c7 TCR, while T102S is a weak agonist and T102G is an antagonist. Hb represents the null peptide, as the MHCII class is the same, but the only interactions between the two is due to nonspecific binding. The data collected demonstrates that cell coupling is both possible and dependent on the affinity of peptide for 5c.c7 TCR in 58α⁻β⁻ cells.

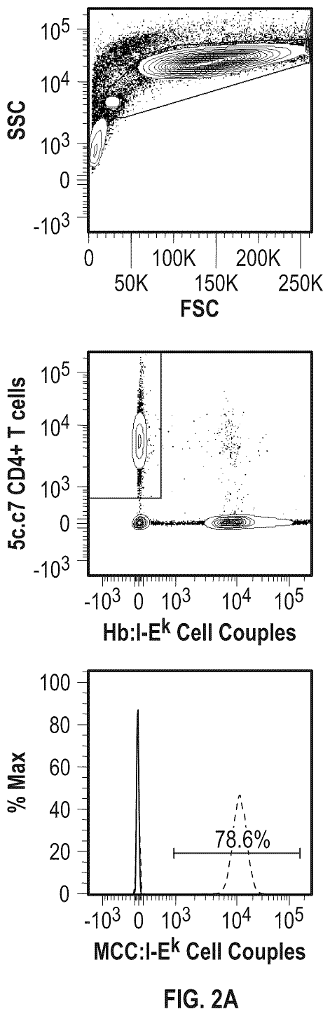

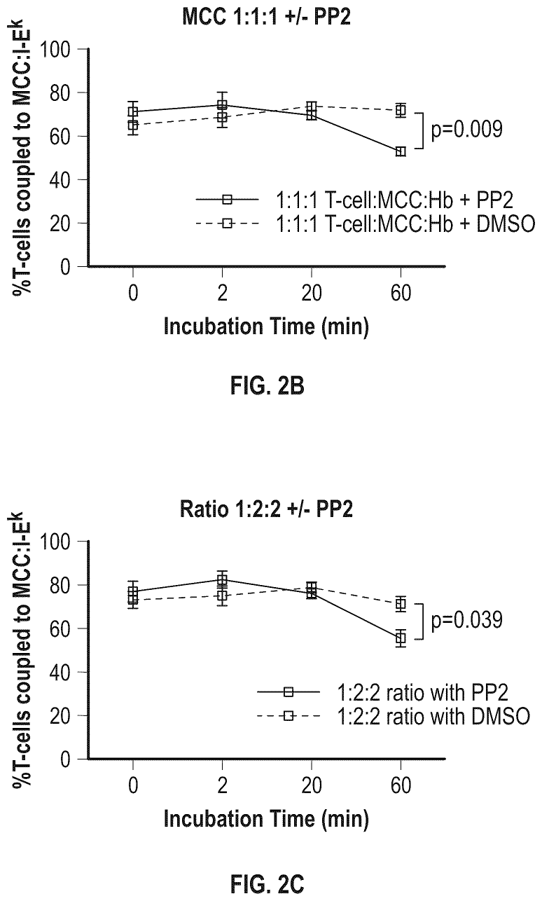

Although cell coupling is possible between 58α⁻β⁻ and M12 cells, we wanted to know if this assay can be applied to naïve mouse cells. Following a similar process in our first experiments, we coupled CFSE labelled CD4+ T cells from a 5c.c7 Rag KO mouse with Tag-it Violet labeled MCC and Hb M12 cells under varying conditions. First, the cells were coupled at a ratio of 1:1:1 or 1:2:2 t-cell:specific APC:dump APC with MCC acting as the specific APC and Hb acting as the dump. A dump APC was used because dump APC should eliminate any nonspecific binding between MCC and the CD4+ T cells. The next variable we analyzed was again the incubation times of 0 minutes, 2 minutes, 20 minutes, and 60 minutes. The third was whether pMHC engagement caused TCR signaling, and consequently, downregulation of TCRs that would inhibit coupling. If the CD4+ cells are indeed downregulating their TCR's as a result of signaling, there should be less coupling observed using flow cytometry. Therefore, a kinase inhibitor, PP2, was introduced which prevents the TCRs from signaling or a DMSO vehicle control. Flow cytometry was used to determine MCC M12 cells coupled to 5c.c7 TCR CD4+ T cells, while excluding Hb-TCR or MCC-Hb-TCR double or triple positive events (FIG. 2A). MCC coupling drastically increased from ˜30% with the hybridomas to anywhere between 60%-85% with the naïve T cells. The 1:1:1 (FIG. 2B) and 1:2:2 (FIG. 2C) ratio saw similar trends in the incubation time and +/−PP2 coupling. With the DMSO vehicle control, cell coupling initially peaked by 2 minutes and slowly decreased until reaching its lowest % by 60 minutes. When PP2 was added, coupling slowly increased in the 1:1:1 ratio, but it increased up to 20 minutes and slightly decreased by the 60-minute mark in the 1:2:2 ratio. In both ratios, the addition of PP2 seems to eliminate TCR downregulation as coupling is statistically higher than with the DMSO control. Although there was not statistical significance between the different ratios +/−PP2 from 0-20 minutes, coupling trended to its highest point by 2 minutes. Previous data suggests that TCR signaling begins quickly (Huse et al., 2007), so this data is consistent with that assertion. It is determined that the optimal parameters to perform CATS is at a 1:1:1 ratio for 2 minutes without PP2.

### Example 3 CATS and Tetramer Analysis of Low Affinity TCR-pMHC Interactions

This example shows how well CATS can pull out antigen specific TCRs within a polyclonal population. Specifically, we wanted to know if the disclosed CATS can be used as or more effectively as tetramers, which represent another antigen-binding method of T cell identification. Tetramers are highly specific, but previous literature suggests that they are ineffective at identifying low affinity TCR-pMHC interactions. In order to determine if CATS could overcome low affinity challenges through its increased avidity, we compared both the cognate pMHCII, MCC:I-E^{K}, M12 cells and MCC tetramer to the lower affinity pMHCII, T102S:I-E^{k}, M12 cells and T102S tetramer. To address this comparison, 1×10⁵ dye-labeled 5c.c7 CD4+ T cells were transferred into a polyclonal B10.A mouse and spleen and lymph nodes were taken 24 hours post-transfer. Flow plots demonstrate how CD4+ T cells were identified when coupled 1:1:1 with MCC or T102S and the dump Hb M12 cells using CATS (FIG. 3A) or how the T cells were identified using MCC:I-E^{k} or T102S:I-E^{k} tetramer while keeping Hb:I-E^{k} tetramer as a dump (FIG. 3B). For further confirmation that our tetramer staining was real, we stained our MCC:I-E^{k} or T102S:I-E^{k} tetramer in 2 colors and gated on the double positive events, excluding any nonspecific binding. We collected the entire sample of cells in order to collect as many dye-labeled T cells that remained in the mouse and compared the percentage of these T cells that were coupled to M12 cells or stained with tetramer (FIG. 3C). Cell coupling with MCC:I-E^{k} and the lower affinity T102S:I-E^{k} M12 cells identified a similar coupling percentage with the dye-labeled T cells (FIG. 3C). And while MCC:I-E^{k} tetramer identified a high percentage of the dye-labeled T cells, T102S:I-E^{k} tetramer failed to identify any weak TCR-pMHC interactions (FIG. 3C). This data demonstrates that CATS can be used to identify weak TCR-pMHC interactions that cannot be identified with tetramer.

### Example 4 Functional Analysis of MHCII in CATS

Based on our results from the immunization experiments, we next asked whether the cell coupling we observed was dependent on the pMHCII molecules themselves or if it was a consequence of nonspecific binding. In this pilot study, spleen and LNs from a naïve and B10.A mouse were taken, and CATS analysis was performed under conditions described below. We saturated the I-E^{k} MHC epitope with 14-4-4S I-E^{k} MHCII antibody, thus blocking any TCR interactions that are dependent on MHC in MCC:I-E^{k}, T102S:I-E^{k}, and Hb:I-E^{k} M12 cells. Furthermore, if these interactions are indeed dependent on MHCII, we would expect coupling to decrease with the addition of the blocking antibody. So, prior to cell coupling, the M12 cells were either left unblocked, stained with an isotype control antibody IgG2a κ, which binds MHCII on epitopes that are different from the 14-4-4S epitope, or stained with MHCII I-E^{k} antibody. 5×10⁵ CD4+ T cells were then coupled with specific APC and dump APC 1:1:1, and cell couples were determined based on flow gating (FIG. 4A). Since the MHCII antibodies are colorless, we wanted to determine that blocking with 20 μg/mL fully saturated the M12 cells. To approach this question, M12 cells were stained with the unconjugated 14-4-4S antibody for 15 minutes at room temperature, excess antibody was washed out, and stained again this time with conjugated 14-4-4S antibody. If the MHCII epitopes of interest were saturated, we would expect to see no staining from the conjugated antibody, which is precisely what occurred (FIG. 4C). We then collected 10,000 events of T cells coupled to our specific APC and determined coupling based on flow gates (FIG. 4B). Cell coupling was then normalized to the blocked cell coupling by dividing the coupling percentage per cell line by its coupling percentage in its blocked state (FIG. 4D). We saw nearly a 4× decrease in coupling between the B10.A CD4+ T cells and MCC and Hb when blocked with the 14-4-4S antibody, and we observed a 3× decrease in coupling between the B10.A CD4+ T cells and T102S (FIG. 4E). These data suggest that the coupling we observe in endogenous polyclonal populations is likely not a consequence of random, nonspecific events. Rather, the data suggest that MHCII is integral to engagement of TCR's with respect to the CATS method, especially regarding the I-E^{k} epitope.

### REFERENCES

The references listed below, as well as those cited in the specification, are hereby incorporated by reference into this disclosure.
Altman J D, Moss P A, Goulder P J, Barouch D H, McHeyzer-Williams M G, Bell J I, McMichael A J, Davis M M. Phenotypic analysis of antigen-specific T lymphocytes. Science. 1996; 274(5284):94-6. PubMed PMID: 8810254.
Corse E, Gottschalk R A, Krogsgaard M, Allison J P. Attenuated T cell responses to a high-potency ligand in vivo. PLOS Biol. 2010; 8(9). Epub 2010 Sep. 22. doi: 10.1371/journal.pbio.1000481.PubMed PMID: 20856903; PMCID: 2939023.
Deshpande N R, Parrish H L, Kuhns M S. Self-recognition drives the preferential accumulation of promiscuous CD4(+) T-cells in aged mice. eLife. 2015; 4. doi: 10.7554/eLife.05949. PubMed PMID: 26173205; PMCID: PMC4501121.
Glassman C R, Parrish H L, Lee M S, Kuhns M S. Reciprocal TCR-CD3 and CD4 Engagement of a Nucleating pMHCII Stabilizes a Functional Receptor Macrocomplex. Cell Rep. 2018; 22(5):1263-75. doi: 10.1016/j.celrep.2017.12.104. PubMed PMID: 29386113; PMCID: PMC5813697.
Huppa J B, Axmann M, Mortelmaier M A, Lillemeier B F, Newell E W, Brameshuber M, Klein L O, Schutz G J, Davis MM. TCR-peptide-MHC interactions in situ show accelerated kinetics and increased affinity. Nature. 2010; 463(7283):963-7. doi: 10.1038/nature08746. PubMed PMID: 20164930; PMCID: 3273423.
Huse M, Klein L O, Girvin A T, Faraj J M, Li Q J, Kuhns M S, Davis M M. Spatial and temporal dynamics of T cell receptor signaling with a photoactivatable agonist. Immunity. 2007July; 27(1):76-88. doi: 10.1016/j.immuni.2007.05.017. Epub 2007 Jul. 12. PMID: 17629516.
Li Y, Yin Y, Mariuzza R A. Structural and biophysical insights into the role of CD4 and CD8 in T cell activation. Frontiers in Immunology. 2013. doi: 10.3389/fimmu.2013.00206
Martinez R J, Andargachew R, Martinez H A, Evavold B D. Low-affinity CD4+ T cells are major responders in the primary immune response. Nat Commun. 2016; 7:13848. doi: 10.1038/ncomms13848. PubMed PMID: 27976744; PMCID: PMC5234832.
Matsui K, Boniface J J, Reay P A, Schild H, Fazekas de St Groth B, Davis M M. Low affinity interaction of peptide-MHC complexes with T cell receptors. Science. 1991. Doi: 10.1126/science.1696397. PubMed PMID: 1696397.
Mogensen T H. Pathogen recognition and inflammatory signaling in innate immune defenses. Clin Microbiol Rev. 2009 April; 22(2):240-73, Table of Contents. doi: 10.1128/CMR.00046-08. PMID: 19366914; PMCID: PMC2668232.
Moon J J, Chu H H, Pepper M, McSorley S J, Jameson S C, Kedl R M, Jenkins M K. Naive CD4 (+) T cell frequency varies for different epitopes and predicts repertoire diversity and response magnitude. Immunity. 2007; 27(2):203-13. Epub 2007 Aug. 21. doi: S1074-7613(07)00366-4 [pii] 10.1016/j.immuni.2007.07.007. PubMed PMID: 17707129.

Parrish H L, Deshpande N R, Vasic J, Kuhns M S. Functional evidence for TCR-intrinsic specificity for MHCII. Proc Natl Acad Sci U S A. 2016. doi: 10.1073/pnas.1518499113.PubMed PMID: 26831112.

1. A system for detecting activation of T cell receptor (TCR), comprising a plurality of cells expressing a major histocompatibility complex (MHC) molecule and a peptide, wherein the peptide is tethered to the MHC to form an MHC-peptide complex, the MHC-peptide complex being presented on surface of said cell, wherein the copy number of the MHC-peptide complex ranges between 10³ and 10⁷, or between 10⁴ and 10⁶ or about 10⁵ per cell.

2. The system of claim 1, wherein the MHC molecule is an MHC class II molecule (MHCII).

3. The system of claim 2, wherein the plurality of cells is derived from a cell line that is capable of perpetuating indefinitely.

4. The system of claim 3, wherein the plurality of cells is derived from a lymphoma cell line.

5. The system of claim 3, wherein the plurality of cells is derived from M12 cells.

6. The system of claim 3, wherein the plurality of cells is M12 cells expressing full-length I-E^{k} MHC.

7. The system of claim 2, wherein the peptide is derived from a foreign pathogen.

8. The system of claim 1, wherein the peptide is derived from a tumor cell and the MHC is an MHC class I.

9. The system of claim 1, wherein the peptide is selected from a library comprising a plurality of peptides that are randomly synthesized.

10. The system of claim 1, wherein the peptide is 10-50 amino acids long, or 10-20amino acids long, or 12-18 amino acids long.

11. The system of claim 1, further comprising a second cell, the second cell comprising a TCR.

12. The system of claim 1, wherein the second cell is 58α⁻β⁻ cell or a primary CD4 T cell.

13. The system of claim 11, wherein the K_{D} between the TCR and the peptide is greater than 10⁻⁶M, or greater than 10⁻⁵M.

14. The system of claim 1, wherein the peptide is tethered to the MHCII through a linker comprising an amino acid sequence of S-G-G-G-G-S.

15. A method for detecting activation of a T cell receptor (TCR), the method comprising (a) contacting a T cell comprising a TCR with a plurality of cells expressing an MHC molecule and a peptide, and (b) determining association between the T cell and the plurality of cells expressing the MHC molecule and the peptide, wherein the peptide is tethered to the MHC to form an MHC-peptide complex, the MHC-peptide complex being presented on surface of said cell, wherein the copy number of the MHC-peptide complex ranges between 10³ and 10⁷, or between 10⁴ and 10⁶ or about 10⁵ per cell.

16. The method of claim 15, wherein step (b) is performed by flow cytometry.

17. The method of claim 15, wherein the T cell and the plurality of cells expressing the MHC molecule and the peptide are labeled by different dyes.
